(19) United States
(12) Patent Application Publication
(10) Pub. No.: US 2026/0257058 A1
(43) Pub. Date: Sep. 3, 2026

(54) ELECTROMEDICAL ELECTRODE ASSEMBLY

(71) Applicant: OSYPKA AG, Rheinfelden-Baden (DE)

(72) Inventors: Peter OSYPKA, Grenzach-Wyhlen (DE); Nicola OSYPKA, Wörthsee (DE); Achim KITSCHMANN, Basel (CH); Francesco SCIUTO, Schopfheim (DE)

(73) Assignee: OSYPKA AG, Rheinfelden-Baden (DE)

(21) Appl. No.: 18/858,363

(22) PCT Filed: Apr. 12, 2023
(86) PCT No.: PCT/EP2023/059541

§ 371 Date: Oct. 21, 2024

(30) Foreign Application Priority Data
Apr. 21, 2022 (DE) 10 2022 109 680.7

(51) Int. Cl. A61N 1/05; A61N 1/39

(52) U.S. Cl. CPC A61N 1/0563; A61N 1/3956

(57) ABSTRACT

The invention relates to improvements in the technical field of electromedical electrode assemblies. As an improvement, an electromedical electrode assembly (1) is proposed which has at least one implantable cardioversion electrode (2) which consists of a flexible, electrically conductive flat structure (3) which is flat in the undeformed position of use and which is designed for flat contact with a target tissue.

### CROSS-REFERENCE TO RELATED APPLICATIONS

This application is a 371 National Phase of PCT/EP2023/059541, filed Apr. 12, 2023, which claims priority from German Patent Application No. 10 2022 109 680.7, filed Apr. 21, 2022, both of which are incorporated herein by reference as if fully set forth.

### TECHNICAL FIELD

The invention relates to an electromedical electrode assembly having at least an implantable cardioversion electrode for delivery of electrical impulses and cardioversion impulses to a target tissue, in particular to heart muscle tissue.

Such electromedical electrode assemblies are implanted temporarily in patients for example after a heart operation and used to support the cardiac activity. Via the implantable cardioversion electrode it is possible to deliver cardioversion impulses to the heart of the patient in case of need in order to correct conduction disturbances. In comparison to pacing or pacemaker impulses, which are delivered to set a certain heart rhythm or to support it, such cardioversion impulses are stronger. To avoid injury to the target tissue, no cardioversion impulses may be delivered via electrodes which are intended exclusively for the delivery of pacemaker impulses.

Due to their strength, cardioversion impulses are also often perceived as painful by patients. This is particularly the case if the cardioversion impulses are delivered via external electrodes. However, cardioversion impulses also delivered via implantable cardioversion electrodes can be perceived by patients as unpleasant, if not even as painful. A further difficulty with the delivery of cardioversion pulses via implantable cardioversion electrodes is to stimulate the target tissue sufficiently for a successful cardioversion. The contact of the cardioversion electrode to the target tissue can have an important influence on the effectiveness of the cardioversion impulses.

### SUMMARY

The object of the invention is to provide an electrode assembly of the type mentioned at the beginning with improved usage properties.

To solve the object, an electromedical electrode assembly having at least one implantable cardioversion electrode for delivering cardioversion pulses to a target tissue, in particular to heart muscle tissue, is proposed, which has the means and features of the independent claim directed to such an electrode assembly. To solve the object in the electrode assembly defined at the beginning, it is thus proposed in particular that the cardioversion electrode of the electromedical electrode assembly consists of a flexible, electrically conductive flat structure which is flat in the undeformed position of use and is designed for flat contact with a target tissue.

Such a flat structure can be applied particularly reliably to the target tissue of the heart to be treated. The flat structure can be placed in the body of the patient to be treated, for example on the outside of the heart, for example on the outside of an atrium of the heart to be treated. In this case, the flat structure can make comparatively extensive contact with the target tissue to be treated and can be reliably applied to the generally curved surfaces of the target tissue to be treated. In this way, sufficient contact can be established between the cardioversion electrode of the electrode assembly and the tissue to be treated for gentle and safe cardioversion. On the one hand, this promotes the effectiveness of the cardioversion treatment and, on the other hand, the large-area contact allows cardioversion pulses to be delivered over a comparatively large area. As a result, the flat structure can promote cardioversion with cardioversion pulses of lower intensity. Cardioversion pulses of lower intensity are generally perceived as less painful and often cause fewer side effects. Thus, in the best case, pain during cardioversion treatment can be reduced or even completely avoided. Overall, the electrode assembly according to the invention thus promotes effective and simultaneously gentle cardioversion by means of an implanted cardioversion electrode. Furthermore, the electrode assembly according to the invention is characterized by its simplified handling and application on the patient. The electrode assembly can be easily applied and allows the use of an electromedical pulse generator, namely a pacer and/or cardioverter at the patient's bedside.

Due to the fact that the cardioversion electrode of the electrode assembly consists of a flexible flat structure, it is also possible to gently explant the cardioversion electrode, for example by pulling on a proximal end of the cardioversion assembly. During explantation, the flat structure can be stretched, for example by pulling on the proximal end of the electrode assembly, and thereby deformed in such a way that an expansion of the flat structure transverse to an explantation direction is reduced during explantation. This promotes atraumatic explantation of the cardioversion electrode.

In an embodiment of the electrode assembly, the flat structure has an electrical resistance of less than 150 ohms, preferably of less than 70 ohms. This can promote the delivery of sufficiently strong cardioversion pulses and protect the target tissue to be treated from injury.

The flat structure can have a longitudinal axis, in particular a longitudinal central axis, which extends in a plane when the flat structure is undeformed. The flat structure is therefore flat in its original undeformed position, but due to its flexibility it can be deformed in such a way that it can be placed flat against a target tissue to be treated.

In one embodiment of the electrode assembly, the flat structure can consist of a longitudinal structure, for example of an electrical conductor, with alternating bending direction and/or be sinusoidal. The longitudinal structure can be an electrical conductor, for example a stranded wire. The conductor can be bent, for example sinusoidally bent to form the flat structure of the cardioversion electrode. In particular in the area of the flat structure of the cardioversion electrode, the conductor is preferably not insulated, so that a delivery of cardioversion pulses via the the conductor forming the flat structure is possible.

Because the flat structure can be sinusoidal and/or consist of a longitudinal structure, for example a conductor that is bent in alternating directions, the flat structure can not only be flexible but also elastic. The elasticity of the flat structure can promote reliable contact between the flat structure and a target tissue.

In a preferred embodiment of the electrode assembly, the flat structure is formed from exactly one electrical conductor. However, it is also possible that the flat structure is formed from several electrical conductors for delivery of cardioversion pulses.

The exact one or at least one conductor from which the flat structure can be formed can have an electrical resistance that is less than 150 ohms, preferably less than 70 ohms. In this way, a flat structure with an electrical resistance of less than 150 ohms, preferably less than 70 ohms, can be formed from the conductor.

The conductor can have a longitudinal axis, in particular a longitudinal central axis, which extends in a plane, for example in a central plane, of the flat structure and/or with an alternating bending direction when the flat structure is undeformed. In this way, the electrical conductor can be used to provide a flat structure with a shape that can be described as a zigzag shape. In one embodiment of the electrode assembly, the electrical conductor can be bent with an alternating bending direction towards the flat structure. The alternating bending direction of the electrical conductor can give the flat structure its flexibility and/or elasticity. The conductor can be bent sinusoidally to form a sinusoidal flat structure.

The electrical conductor may have a diameter D of for example 0.1-0.5 mm. In one embodiment, the conductor may have a diameter D of 0.1-0.3 mm, for example a diameter D of 0.2 mm.

The electrical conductor may have at least two adjacent and/or interconnected conductor sections, which together span an angle a between 10° and 170°. The conductor may have at least two adjacent conductor sections, which together span an angle a between 30° and 90° or also an angle a between 30° and 60° or also an angle a of 35°.

Two adjacent conductor sections of the conductor can be connected to each other by a bending radius of, for example, 0.3 mm to 2 mm.

In the unstretched state, the flat structure can have a length extension L1 measurable in the direction of its longitudinal axis of between 10-50 mm inclusive in each case, in particular between 25-40 mm inclusive in each case. In the unstretched state, the flat structure can have a measurable extension L2 transverse to its longitudinal axis of 5-30 mm inclusive in each case and/or be formed from at least one conductor whose length is 40 -140 mm.

The flat structure can have a stripped stranded wire as at least one electrical conductor for emitting cardioversion pulses.

The flat structure, in particular the electrical conductor, can be made of stainless steel, platinum, a platinum alloy, gold, a gold alloy, magnesium, a magnesium alloy, molybdenum or a molybdenum alloy, for example. The use of stainless steel is particularly preferred, as this material has been tried and tested on patients for decades, has been found to be harmless to humans and is also particularly suitable for the application of producing the flat structure described above due to its resilience, flexibility and elasticity.

In a particularly preferred embodiment of the electrode assembly, this has two fixing means between which the flat structure is arranged. The fixing means of the electrode assembly are set up for fixing and/or clamping of the flat structure to a target tissue, in particular to an outer side of the heart. With the aid of the two fixing means of the electrode assembly, the flat structure can be brought as cardioversion electrode onto an outer side of a target tissue to be treated in contact and held there. In particular in the case that the flat structure is not only flexible but also elastic, the flat structure can be stretched in its position of use on a target tissue to be treated with the aid of the two fixing means. Due to its elasticity, the flat structure can then automatically apply itself to the target tissue against the retaining force of the two fixing means and keep it applied, thus ensuring reliable contact with the target tissue to be stimulated. The two fixing means thus promote the reliable contact of the flat structure of the electrode assembly, which serves as a cardioversion electrode, with a beating heart. The flat contact of the flat structure under tension can prevent the detachment of the flat structure or even only parts of the flat structure. This promotes reliable and gentle cardioversion treatment using the electrode assembly.

In one embodiment of the electrode assembly, the flat structure has a distance of between 10 and 70 mm from at least one of the two fixing means or from both fixing means. As previously mentioned, the flat structure can be clamped to a target tissue and/or adapted to an atrial size of a heart to be treated, in particular by the previously mentioned fixing means. When stretched, the flat structure can cover an area of between 30 mm² and 1600 mm², for example. The area covered by the flat structure can be calculated from the product of the longitudinal extension and the transverse extension of the stretched flat structure.

A distal fixing means of the electrode assembly for fixing and/or clamping the flat structure can be arranged distally of the flat structure. For example, the flat structure can be arranged at a distal end of the electrode assembly or between a distal end of the electrode assembly and the flat structure.

In principle, it is possible to position the fixing means with suture material in the target tissue in order to fix the fixing means in the target tissue and thus anchor the electrode assembly. However, this can be comparatively time-consuming, as the suture material must first be connected to the fixing means.

In order to avoid this effort, the electrode assembly can have at least one positioning means at its distal end, for example a heart needle, for positioning a fixing means, in particular the aforementioned fixing means, in the target tissue. The positioning means can be used to position the fixing means for anchoring the target tissue there. After successful positioning, the positioning means can be detached and removed.

The electrode assembly can have a connecting lead via which the flat structure can be connected to a stimulation generator. For this purpose, the electrode assembly can have a connection means that is arranged or formed at the proximal end of the connecting lead. The connecting lead can be connected to the flat structure at a point that lies on a longitudinal axis, in particular on a longitudinal central axis of the flat structure.

The connecting lead can be used as a pulling means, with the aid of which the flat structure acting as a cardioversion electrode can be removed from its implanted position of use by pulling. If the connecting lead is connected to the flat structure at a point that lies on a longitudinal central axis of the flat structure, the flat structure can be deformed by the traction during explantation in such a way that a cross-sectional reduction of the flat structure, preferably uniform and aligned transversely to the direction of traction, is obtained. This can promote atraumatic explantation of the flat structure.

In one embodiment of the electrode assembly, it is provided that it has at least one lateral electrode for emitting pacing pulses, which can also be referred to as pacemaker pulses.

For example, in conjunction with the cardioversion electrode, the lateral electrode can also be used to deliver pacing pulses to the heart to be treated. The electrode assembly can therefore take on a dual function and deliver not only cardioversion impulses but also pacing impulses to the heart.

The at least one lateral electrode of the electrode assembly can be assigned at least one fixing means for fixing the lateral electrode to or in a target tissue. In this way, it is possible to reliably anchor the lateral electrode in the target tissue for the time during which the electrode assembly is to be used to support the cardiac function of a patient.

In a particularly advantageous embodiment of the electrode assembly, it is provided that the fixing means associated with the lateral electrode is a fixing means of at least two fixing means for fastening and/or clamping the flat structure.

In this way, the fixing means assigned to the lateral electrode thus has a dual function. On the one hand, the fixing means is used to anchor the lateral electrode in the target tissue and, on the other hand, it also serves to reliably hold the flat structure, which serves as a cardioversion electrode, in its position of use. In this embodiment of the electrode assembly, it is therefore possible to dispense with a separate fixing means that only serves to fix the lateral electrode.

For reliable fixing of the lateral electrode and the cardioversion electrode, namely the flat structure, only a minimum of fixing means is provided in this embodiment of the electrode assembly. In this way, lesions on the target tissue caused by the fixing means are kept to a minimum.

The at least one lateral electrode and the at least one cardioversion electrode can be arranged at a freely selectable angle relative to each other. In a preferred embodiment of the electrode assembly, the lateral electrode and the cardioversion electrode together span an angle of between 1° and 170°. In one embodiment of the electrode assembly, the aforementioned connecting lead, the lateral electrode and the cardioversion electrode can together form a Y-shape in the implanted position of use.

The electrode assembly, in particular the at least one cardioversion electrode and/or the at least one lateral electrode, can be explantable by pulling on a proximal end of the electrode assembly, in particular on a proximal end of the previously mentioned connecting lead.

The conductor of which the flat structure can consist can be an electrical conductor of the connecting lead. The flat structure can therefore consist of an electrical conductor, in particular a stranded wire, of the connecting lead.

### BRIEF DESCRIPTION OF THE DRAWINGS

In the following exemplary embodiments of the invention are described in more detail with reference to the drawing. The invention is here not limited to the shown exemplary embodiments. Further exemplary embodiments of the invention result from by combination of the features of the claims and/or by combination of the features of the exemplary embodiments shown in the figures, wherein:

FIG. 1: shows a side view of a first embodiment of an electrode assembly with an implantable cardioversion electrode, which consists of a flexible, electrically conductive flat structure which is flat in the undeformed initial position and is designed for flat contact with a target tissue,

FIG. 2: shows a variant of the electrode assembly shown in FIG. 1, having a lateral electrode in addition to the cardioversion electrode, via which pacing pulses can be delivered to a target tissue, and

FIG. 3: shows an enlarged detailed view of the flat structure of the electrode assemblies shown in FIGS. 1 and 2.

### DETAILED DESCRIPTION

All figures show at least parts of electromedical electrode assemblies designated as a whole with 1. Each electrode assembly 1 shown is designed to deliver electrical impulses to a target tissue, namely to heart muscle tissue. Each electrode assembly 1 shown has an implantable cardioversion electrode 2 for delivering cardioversion pulses.

The cardioversion electrodes 2 each consist of a flexible, electrically conductive flat structure 3 which is flat in its undeformed initial position and is designed to lie flat against a target tissue. The flat structure 3 is elastic and has an electrical resistance of less than 150 ohms, preferably less than 70 ohms.

The flat structure 3 has a longitudinal axis 4, namely a longitudinal central axis, which extends in a plane when the flat structure 3 is undeformed. The flat structure 3 is formed from a longitudinal structure, namely an electrical conductor 5 with an alternating bending direction and is therefore zigzag-shaped. In the exemplary embodiments of flat structures 3 shown in the figures, these consist of exactly one electrical conductor 5, which is set up to emit cardioversion pulses.

The conductor 5 has an electrical resistance that is less than 150 ohms, preferably less than 70 ohms. In this way, the conductor 5 can be used to provide a flat structure 3 that has an electrical resistance of less than 150 ohms, preferably less than 70 ohms, which is favorable for cardioversion.

A longitudinal axis of the conductor 5 is arranged in a plane, in particular in a central plane, of the flat structure 3 when the flat structure 3 is undeformed and extends through this central plane with alternating bending direction. In particular, the illustration in FIG. 3 makes it clear that the flat structure 3 is formed from the conductor 5, which is bent with alternating bending directions. The conductor 5 has a diameter D of 0.1-0.5 mm. The diameter of the conductor 5 can be 0.1-0.3 mm or even 2 mm, for example. The conductor 5 has mutually adjacent conductor sections 6 that are connected to each other and together form an angle a of between 10° and 170°.

In the exemplary embodiment shown in FIG. 3, the angle a is approx. 35°. The mutually adjacent conductor sections 6 are connected to each other via a bending radius R. The bending radius R can be between 0.3 mm and 2 mm, for example.

In the unstretched state, the flat structure 3 can, for example, have a length extension L1 measurable in the direction of its longitudinal axis 4 of between 10-50 mm in each case. In the unstretched state, the flat structure has a measurable extension L2 transverse to its longitudinal axis 4 of, for example, 5-30 mm in each case.

The electrical conductor 5, from which the flat structure 3 is made, can have a length of 40 -140 mm, for example. FIG. 3 also indicates a linear expansion L3, which the flat structure 3 can have in a stretched, clamped state. The length extension L3 is greater than the length extension L1 and can be a multiple of the length extension L1 of the flat structure 3 in the unstretched state, for example one and a half times, two times, two and a half times or even three times.

The flat structure 3 has a stripped stranded wire as the electrical conductor 5 for emitting cardioversion pulses. The flat structure 3, namely its electrical conductor 5, is made of stainless steel.

In the second embodiment of the electrode assembly 1 shown in FIG. 2, it can be seen that the electrode assembly 1 has two fixing means 7, 8, between which the flat structure 3 is arranged. The fixing means 7, 8 serve to clamp the flat structure 3 to an outer side of the heart and to fasten it in a clamped manner. The flat structure 3 is at a distance of between 10 and 70 mm from at least one of the two fixing means 7,8.

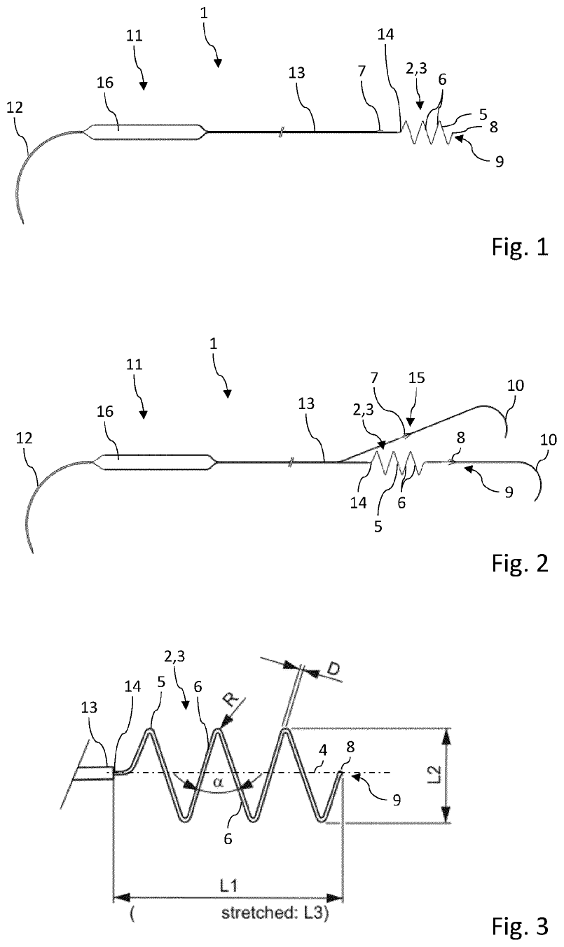

In the electrode assembly 1 shown in FIG. 1, the flat structure 3 is also arranged between two fixing means 7 and 8 of the electrode assembly 1 that are comparable in terms of their function. These fixing means 7 and 8 are also used to attach and/or clamp the flat structure 3 to a target tissue, for example to the outside of the heart.

The flat structure 3 can be clamped by the two fixing means 7 and 8 and can be adapted to an atrial size of a heart to be treated by clamping and stretching the flat structure 3. When clamped, the flat structure 3 can cover an area of between 30 mm² and 1600 mm². This area can correspond to the product of the aforementioned expansions L2 and L3 of the clamped and stretched flat structure 3.

The distal fixing means 8 of the two fixing means 7 and 8 of the electrode assembly 1 is arranged distally of the flat structure 3, for example between a distal end 9 of the electrode assembly 1 and the flat structure 3. At its distal end 9, the electrode assembly 1 has an anchoring means 10 in the form of a heart needle. The anchoring means 10 can be used to anchor the electrode assembly 1 in the target tissue. The positioning means 10 serves to bring the distal fixing means 8 into its position of use in the target tissue. The positioning means 10 can then be detached and removed.

At its proximal end 11, the electrode assembly 1 also has a further needle 12 with which the electrode assembly 1, namely its connecting lead 13, can be pierced through the body wall of a patient and guided outwards.

The flat structure 3 can be connected to a stimulation generator via the connecting lead 13. The connecting lead 13 is connected to the flat structure 3 at a point 14, which is located on the longitudinal central axis 4 of the flat structure 3. Point 14 can also be referred to as the connection point.

The conductor 5 can be an electrical conductor, in particular a stranded wire, of the connecting lead 13. The flat structure 3 can therefore consist of an electrical conductor, in particular a stranded wire, of the connecting lead 13. The previously mentioned point 14 then defines the transition between the connecting lead 13 and the flat structure 3.

At its proximal end 11, each of the electrode assemblies 1 shown has a connection means 16 with which the respective connecting lead 13 can be connected to a stimulation generator. The connection means 16 can be a connector plug, for example.

The electrode assembly 1 shown in FIG. 2 has an implantable lateral electrode 15 in addition to the cardioversion electrode 2. The lateral electrode 15 is used to deliver pacing impulses to the target tissue. Pacing pulses can be delivered in conjunction with the cardioversion electrode 2.

The lateral electrode 15 of the electrode assembly 1 shown in FIG. 2 is associated with the fixing means 7 of the two fixing means 7 and 8. The fixing means 7 is thus also used to fix the lateral electrode 15 in a target tissue. The fixing means 7 assigned to the lateral electrode 15 is thus one of the two fixing means 7 and 8, which is used for fixing and/or clamping the flat structure 3. This fixing means 7 can be introduced into the target tissue with the aid of an anchoring means 10, which is arranged distal to the lateral electrode 15. This positioning means 10 is also designed as a heart needle.

FIG. 2 indicates that the lateral electrode 15 and the at least one cardioversion electrode 2, namely the flat structure 3, can be arranged at a freely selectable angle relative to each other, for example at an angle between 1° and 170°. The arrangement of the lateral electrode 15 and the cardioversion electrode 2 in an angle range of 1° to 170° and preferably at an angle to each other that is less than 90° can promote the gentle and preferably atraumatic explantation of the electrode assembly. The lateral electrode 15, the cardioversion electrode 2 and the connecting lead 13 then form a Y-shape.

The electrode assembly 1, namely the cardioversion electrode 2 and the lateral electrode 15 can be explanted by pulling on the proximal end 11 of the electrode assembly 1, namely by pulling on the proximal end 11 of the connecting lead 13 of the electrode assembly 1. The electrode assembly 1 shown in FIG. 1 can also be explanted in the same way by pulling on the connecting lead 13.

The invention deals with improvements in the technical field of electromedical electrode assemblies. As an improvement, an electromedical electrode assembly 1 is proposed which has at least one implantable cardioversion electrode 2 which consists of a flexible, electrically conductive flat structure 3 which is flat in the undeformed position of use and which is designed for flat contact with a target tissue.

### LIST OF REFERENCE SIGNS

1 Electromedical electrode assembly
2 Cardioversion electrode
3 Flat structure
4 Longitudinal axis of 3
5 Longitudinal structure, electrical conductor
6 Adjacent conductor sections
7 Fixing means
8 Distal fixing means
9 Distal end of 1
10 Positioning device, heart needle
11 Proximal end
12 Needle on 11
13 Connecting lead
14 Point
15 Lateral electrode
16 Connecting means

1. An electromedical electrode assembly (1), comprising:
at least one implantable cardioversion electrode (2) for delivery of cardioversion pulses to a target tissue, tissue,
wherein the cardioversion electrode (2) is formed of a flexible, electrically conductive flat structure (3) which is flat in an undeformed initial position and is adapted for flat contact with a target tissue.

2. The electrode assembly (1) according to claim 1, wherein the flat structure (3) is elastic.

3. The electrode assembly (1) according to claim 1, wherein at least one of a) the flat structure (3) has a longitudinal axis (4) which extends in a plane when the flat structure (3) is undeformed, or b) the flat structure (3) includes a longitudinal structure (5) with at least one of an alternating or sinusoidal bending direction.

4. The electrode assembly (1) according to claim 1, wherein the flat structure (3) is formed from at least one electrical conductor (5) for emitting cardioversion pulses.

5. The electrode assembly (1) according claim 4, wherein the at least one electrical conductor (5) has an electrical resistance which is less than 150 ohms.

6. The electrode assembly (1) according to claim 5, wherein a longitudinal axis of the at least one conductor (5) extends in a plane of the undeformed flat structure (3) or in alternating bending directions, and/or wherein the flat structure (3) is formed from the conductor (5) bent at least one of with alternating bending directions or sinusoidally.

7. The electrode assembly (1) according to claim 6, wherein at least one of a) the at least one conductor (5) has a diameter D of 0.1 to 0.5 mm, b) the conductor (5) has at least two mutually adjacent and/or interconnected conductor sections (6) which together span an angle α between 10° and 170°, or c) the at least two conductor sections (6) are connected to one another via a bending radius (R).

8. The electrode assembly (1) according to claim 1, wherein the flat structure (3) in an unstretched state has at least one of a) a length extension L1 measurable in a direction of a longitudinal axis (4) thereof of between 10 and 50 mm inclusive in each case, or b) an extension L2 measurable transversely to the longitudinal axis (4) of between 5 and 30 mm inclusive in each.

9. The electrode assembly (1) according to claim 1, wherein the at least one electrical conductor (5) is a stripped stranded wire for emitting cardioversion pulses.

10. The electrode assembly (1) according to claim 1, the flat structure (3) is selected from the group consisting of stainless steel, platinum, a platinum alloy, gold, a gold alloy, magnesium, a magnesium alloy, molybdenum or a molybdenum alloy.

11. The electrode assembly (1) according to claim 1, further comprising two fixing means (7, 8) between which the flat structure (3) is arranged and which serve to at least one of fix or clamp the flat structure (3) to the target tissue.

12. The electrode assembly (1) according to claim 11, wherein the flat structure (3) has a distance between 10 and 70 mm inclusive from at least one of the two fixing means (7, 8).

13. The electrode assembly (1) according to claim 1, wherein at least one of a) the flat structure (3) is at least one of stretchable or adaptable to an atrial size of a heart to be treated, in particular by the fixing means (7, 8), or b) the flat structure (3), when stretched, covers an area between 30 mm² and 1600 mm².

14. The electrode assembly (1) according to claim 11, wherein a distal one of the fixing means (8) is arranged distally of the flat structure (3).

15. The electrode assembly (1) according to claim 1, further comprising at least one positioning means (10) at a distal end (9) of the electrode assembly (1) for positioning a fixing means (8) in the target tissue.

16. The electrode assembly (1) according to claim 1, further comprising a connecting lead (13) via which the flat structure (3) is connectable to a stimulation generator, and the connecting lead (13) is connected to the flat structure (3) at a point (14) which lies on a longitudinal central axis (4) of the flat structure (3).

17. The electrode assembly (1) according to claim 1, further comprising at least one implantable lateral electrode (15) for emitting pacing pulses.

18. The electrode assembly (1) according to claim 17, further comprising at least one fixing means (7, 8) for fixing the lateral electrode (15) in a target tissue associated with the at least one lateral electrode (15).

19. The electrode assembly (1) according to claim 18, wherein the at least one fixing means (7, 8) associated with the lateral electrode (15) is a fixing means of at least two fixing means (7, 8) for at least one of fixing or clamping the flat structure (2).

20. The electrode assembly (1) according to claim 17, wherein the at least one lateral electrode (15) and the at least one cardioversion electrode (2) are arrangeable at a freely selectable angle relative to one another.

21. The electrode assembly (1) according to claim 17, wherein at least one of the at least one cardioversion electrode (2) or the at least one lateral electrode (15) is explantable by pulling on a proximal end (11) of the electrode assembly (1).
